(19) United States
(12) Patent Application Publication
(10) Pub. No.: US 2016/0117451 A1
(43) Pub. Date: Apr. 28, 2016

(54) Method For Determining Change In Condition For Soft Tissue Injury

(71) Applicant: Maryrose Cusimano Reaston, Carlsbad, CA (US)

(72) Inventor: Maryrose Cusimano Reaston, Carlsbad, CA (US)

(21) Appl. No.: 14/987,499

(22) Filed: Jan. 4, 2016

Related U.S. Application Data
(63) Continuation-in-part of application No. 13/487,725, filed on Jun. 4, 2012.

(51) Int. Cl. G06F 19/00; A61B 5/11; A61B 5/0488

(52) U.S. Cl. CPC G06F 19/322; G06F 19/345; G06F 19/328; A61B 5/0488; A61B 5/1124

(57) ABSTRACT

A method for determining change in condition for soft tissue injury that utilizes a baseline test and a post-loss test. An individual, such as an employee, consents to having a baseline test performed on them. The results of the baseline test are not read/reviewed at the time of testing, but instead stored in a database along with information pertaining to the individual. When the individual later files a claim for compensation from an injury based on an injury reported, the post-loss test that is related to the baseline test is performed. The post-loss results are then compared with the baseline test results to determine the authenticity and extent of the claimed injury. The baseline test results are not read/reviewed until an injury is alleged, and then only the pertinent baseline test results are utilized, thereby insuring legal compliance.

### TECHNICAL FIELD

The invention generally pertains to medical systems, and more particularly to a method for determining change in condition for soft tissue injury in a manner that maintains objectivity in pre-loss and post-loss assessment of an injury.

### BACKGROUND ART

When a person seeks compensation for an injury, whether the injury incurred on the job, in a car accident, through medical malpractice, or otherwise, it is necessary to determine whether the type of injury is one which is compensable based on relevant rules. The relevant rules could arise from a worker's compensation statute or regulation, a health care policy, legal proceeding, etc. In these situations, the injured individual cannot be forced to undergo an invasive test or functional assessment, such as a CAT or MRI scan, to determine the location and extent of injury.

In addition, laws and regulations increasingly require functional assessments to prevent discrimination in the determination of compensation for an injury. One such example is the Americans with Disabilities Act (ADA), which requires any such testing to be non-invasive, non-loading, and objective. In other words, any such test must be related to the underlying context, such as the body part(s) related to the job description of an injured person. Other regulatory requirements exist and are likely to be promulgated in the future as well.

In the employment context, this places a burden on an employer, who needs to be able to discern between compensable injuries that occur at the workplace, and those pre-existing injuries that are unrelated to and not aggravated by the workplace injury. Under the ADA, it would be discriminatory to require an employee to disclose any existing injuries or conditions as a condition for hire or continued employment.

Similar situations arise in health insurance, sports medicine, the legal fields, and other fields where there are restrictions in what a compensation provider can require to assure that only compensable injuries are being covered by the provider.

Therefore, it would be beneficial to provide a method that can filter out pre-existing injuries unrelated to, or not aggravated by, an injury for which a person is seeking compensation. The method optimally functions in a non-invasive, non-loading, and non-discriminatory manner, and provides specific treatment recommendations for any functional capacity changes caused by an injury(ies).

A search of the prior art did not disclose literature or patents that read directly on the claims of the instant invention. However, the following U.S. patents are considered related.

| PAT. NO. | INVENTOR | ISSUED |
|---|---|---|
| 8,117,047 | Cusimano-Reaston et al | 14 Feb. 2012 |

The U.S. Pat. No. 8,117,047 patent discloses a business model for a healthcare provider organization (HPO) which comprises a preferred provider network or other membership agreement that allows individuals or groups to join via a membership contract. The contract allows the HPO to provide, a technical component of a medical evaluation or Service. Additionally, the HPO employs or retains the services of healthcare professionals who participate in and monitor an evaluation of a patient who can be at a remote location form the healthcare professional. The HPO provides a medical diagnostic unit that allows the healthcare professional to receive data that pertains to the patient via a real-time communication protocol or the patient data is collected and stored on an electronic storage device. The healthcare professional then analyzes the patient data and issues recommended treatment.

For background purposes and as indicative of the art to which the invention relates, reference may be made to the following remaining patents found in the search.

| PAT. NO. | INVENTOR | ISSUED |
|---|---|---|
| 5,163,440 | DeLuca et al | November 1992 |
| 5,361,775 | Remes et al | November 1994 |
| 5,368,043 | Sunouchi et al | November 1994 |
| 5,462,065 | Cusimano | October 1995 |
| 5,513,651 | Cusimano, et al | May 1996 |
| 5,585,785 | Gwin et al | December 1996 |
| 5,662,118 | Skubick | September 1997 |
| 5,722,420 | Lee | March 1998 |
| 5,755,675 | Sihvonen | May 1998 |
| 5,784,635 | McCallum | July 1998 |
| 5,785,666 | Costello et al | July 1998 |
| 5,919,148 | Marko et al | July 1999 |
| 5,964,719 | Costello et al | October 1999 |
| 6,004,312 | Finneran et al | December 1999 |
| 6,159,147 | Lichter et al | December 2000 |
| 6,185,451 | Richardson et al | February 2001 |
| 6,265,978 | Atlas | July 2001 |
| 6,280,395 | Appel et al | August 2001 |
| 6,322,502 | Schoenberg et al | November 2001 |
| 6,352,516 | Pozos et al | March 2002 |
| 6,440,067 | DeLuca et al | August 2002 |
| 6,441,747 | Khair et al | August 2002 |
| 6,439,578 | DeFeo | December 2002 |
| 6,496,705 | Ng et al | December 2002 |
| 6,575,902 | Burton | June 2003 |
| 6,597,944 | Hadas | July 2003 |
| 6,631,297 | Mo | October 2003 |
| 6,643,541 | Mok et al | November 2003 |
| 6,647,288 | Madill et al | November 2003 |
| 6,678,549 | Cusimano et al | January 2004 |
| 6,738,798 | Ploetz et al | May 2004 |
| 6,745,062 | Finneran et al | June 2004 |
| 6,816,603 | David, et al | November 2005 |
| 6,856,833 | Finneran et al | February 2005 |
| 6,915,148 | Finneran et al | July 2005 |
| 6,917,825 | Finneran et al | July 2005 |
| 6,917,829 | Kwong | July 2005 |
| 6,965,794 | Brody | November 2005 |
| 7,020,508 | Stivoric et al | March 2006 |
| 7,027,621 | Prokoski | April 2006 |
| 7,058,438 | Grace et al | June 2006 |
| 7,127,279 | Finneran et al | October 2006 |
| 7,130,673 | Tolvansen-Laakso et al | October 2006 |
| 7,150,715 | Collura et al | December 2006 |
| 7,160,253 | Nissila | January 2007 |
| 7,188,151 | Kumar et al | March 2007 |
| 7,313,957 | Kuramori et al | January 2008 |
| 7,363,069 | Finneran et al | April 2008 |
| 7,381,185 | Zhirnov et al | June 2008 |
| 7,409,242 | Mackawa et al | August 2008 |
| 7,421,293 | Kuramori et al | September 2008 |
| 7,433,732 | Carney et al | October 2008 |
| 7,467,010 | Kuramori et al | December 2008 |
| 7,486,987 | Kuramori et al | February 2009 |
| 7,493,157 | Gozani et al | February 2009 |
| 7,532,925 | Kuramori et al | May 2009 |
| 7,574,369 | Borza | August 2009 |
| 7,602,301 | Stirling et al | October 2009 |
| 7,627,358 | Finneran et al | December 2009 |
| 7,628,761 | Gozani et al | December 2009 |
| 7,634,315 | Cholette | December 2009 |
| 7,649,445 | Kuramori et al | January 2010 |
| 7,693,572 | Kuramori et al | April 2010 |
| 7,725,175 | Koeneman et al | May 2010 |
| 7,764,990 | Martikka et al | July 2010 |
| 7,767,149 | Maus et al | August 2010 |
| 7,809,435 | Ettare et al | October 2010 |
| 7,831,302 | Thomas | November 2010 |
| 7,860,562 | Endo et al | December 2010 |
| 7,912,526 | Finneran et al | March 2011 |
| 7,970,734 | Townsend et al | June 2011 |
| 2005/0065819 | Schultz | March 2005 |
| 2005/0251421 | Chang et al | November 2005 |
| 2006/0155173 | Anttila et al | July 2006 |
| 2006/0190300 | Drucker | August 2006 |
| 2006/0265255 | Williams | November 2006 |
| 2006/0271445 | Lee et al | November 2006 |
| 2007/0016098 | Kim et al | January 2007 |
| 2007/0027369 | Pagnacco et al | February 2007 |
| 2007/0027388 | Chou | February 2007 |
| 2007/0033070 | Beck et al | February 2007 |
| 2007/0130287 | Kumar et al | June 2007 |
| 2007/0270665 | Yang et al | November 2007 |
| 2008/0058668 | Seyed Momen et al | March 2008 |
| 2009/0299210 | Marcarian | December 2009 |
| 2010/0106044 | Linderman | April 2010 |
| 2010/017735 | Hoppe | June 2010 |

### DISCLOSURE OF THE INVENTION

The invention discloses a method for determining change in condition for injury soft tissue. In its basic design, the method utilizes a computer server and a memory medium, and is comprised of the following steps:
1) have an employee sign a consent from,
2) receive a specific job description from an employer for the employee in a specific job position,
3) generate a baseline test which comprises a testing protocol based on the employee's specific job description,
4) perform a baseline test on the employee,
5) acquire baseline test results for the employee in the specific job position,
6) store in the memory medium, without interpretation, the baseline test results for the employee in the specific job position,
7) perform a post-loss test on the employee after an injury is alleged, wherein the post-loss test is specific to the alleged injury of the employee,
8) acquire post-loss test results,
9) store in the memory medium the post-loss test results, and
10) compare the post-loss test results with the baseline test results to determine a change in condition for soft tissue injury between the post-loss test results and the baseline test results and to record a comparison result.

The method utilizes hardware, firmware, software, or a combination thereof. The method may also be implemented as instructions stored on the memory medium, which may be read and executed by one or more processors. The memory medium may also include any mechanism for storing or transmitting information. For example, the memory medium may include storage media such as read only memory (ROM), random access memory (RAM), magnetic disk storage media, optical storage media, and flash memory devices; transitory media such as electrical, optical, acoustical or other forms of propagated signals (e.g., carrier waves, infrared signals, digital signals, etc.). Further, firmware, software, routines, and/or instructions may be described herein as performing certain actions. However, it should be noted that such descriptions are merely for convenience and that such actions in fact result from computing devices, processors, controllers, or other devices executing the firmware, software, routines, instructions, etc.

In view of the above disclosure, the primary object of the invention is to provide a method for determining change in condition for soft tissue injury that is capable of maintaining objectivity in pre-loss and post-loss assessment of an injury(ies) and that remains in an un-read state until an injury is alleged, at which time only the test results pertaining to the specific alleged injury(ies) are compared, thereby insuring compliance with current law.

In addition to the primary object, it is also an object of the invention to provide a method for determining change in condition for soft tissue injury that:
utilizes steps that are easy to learn and perform,
can be utilized for various purposes including health insurance, sports medicine or legal fields,
is ADA compliant,
is non-invasive,
can be used at various locations and for various muscle groups on a person's body,
is legally admissible,
can significantly reduce the number of fraudulent worker compensation claims/payments,
is non-discriminatory and
is cost effective from an employer's, employee's and health care provider's point of view.

These and other objects and advantages of the present invention will become apparent from the subsequent detailed description of the preferred embodiment and the appended clams taken in conjunction with the accompany drawings.

### BRIEF DESCRIPTION OF THE DRAWING

FIGS. 1A and 1B is a block diagram of steps performed in a method for determining change in condition for soft tissue injury.

### BEST MODE FOR CARRYING OUT THE INVENTION

The best mode for carrying out the invention is presented in terms that disclose a method for determining change in condition for soft tissue injury. The method determines if an injury results from the normal actions/activities that are performed during the course and scope of a particular employment. The method can also determine optimal treatment for an injury that has occurred. The method utilizes proprietary software and is designed to maintain objectivity in pre-loss and post-loss assessment by utilizing non-loading, non-invasive functional assessments that do not identify an individual's personal data or disability, and provides a pre-injury “snapshot” that is only reviewed/compared after an injury assessment. The method does not identify any disability on a person, such as an employee who alleges a work place injury, since such identification is illegal.

It is important to note that test data which is acquired and stored is never read/reviewed by a human, since such an action is illegal. Any reading/reviewing of the test data by a human would render the test results un-useable, therefore resulting in the entire method being un-viable.

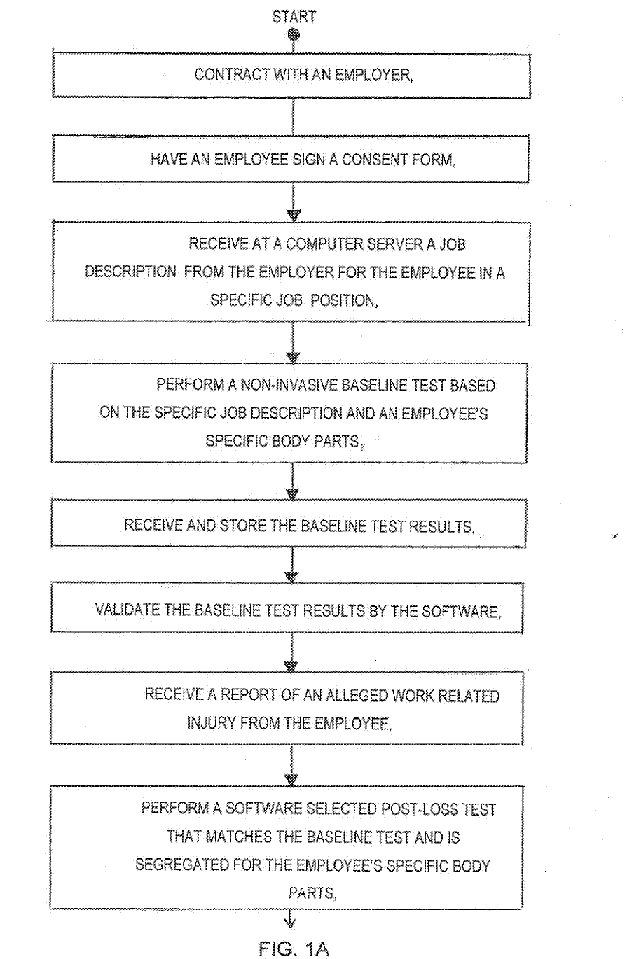

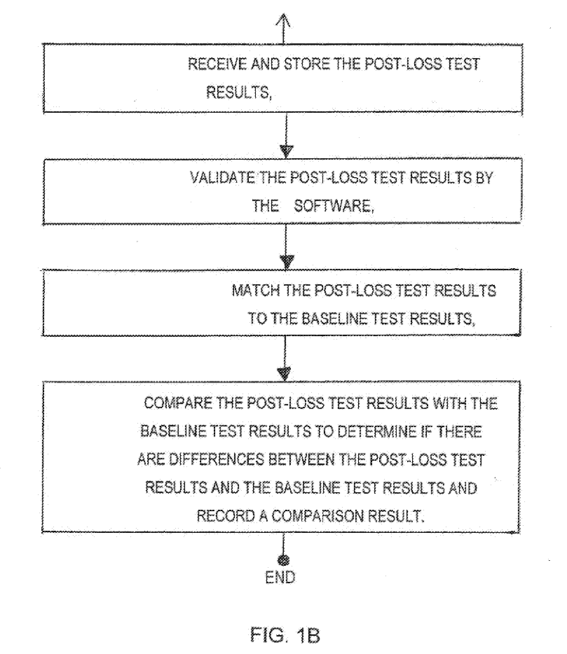

To utilize the method, the following steps, as shown in FIGS. 1A and 1B, are performed:
a) contract with an employer,
b) have an employee sign a consent form,
c) receive at a computer server a job description from the employer for the employee in a specific job position,
d) perform a non-invasive baseline test based on the specific job description and an employee's specific body part(s),
e) receive and store the baseline test results,
f) validate the baseline test results by the software,
g) receive a report of an alleged work related injury from the employee,
h) perform a software selected post-loss test that matches the baseline test and is segregated for the employee's specific body parts,
i) receive and store the post-loss test results,
j) validate the post-loss test results by the software,
k) match the post-loss test results to the baseline test results, and
l) compare the post-loss test results with the baseline test results to determine if there are differences between the post-loss test results and the baseline test results and to record a comparison result.

The method utilizes two tests, or testing protocols: a baseline test and a post-loss test. The method is designed to optimally be utilized by employers and employees, although other applications that involve allegations of injury that must be determined or proven can also utilize the method.

Initially, an employer will have an employee, either an existing employee or a recently-hired employee, sign a consent form to allow the tests to be performed and the method to be utilized if or when it becomes necessary. For many types of employment, the signing of a consent form will be company policy and a condition for continuing or new employment. Typically, an employee should not have any issues or concerns relating to the signing of the consent form since the baseline test results are not read/reviewed and are non-invasive, thereby insuring that the employees' legal rights are upheld and respected.

As previously disclosed, no human can or will ever read/review the test results since doing so is illegal. A human cannot perform this action, only a computer. Human interaction with the test results would result in the entire method being un-useable and un-viable for the required purpose.

The baseline test constitutes part of a soft tissue management program which also includes the post-loss test and other features that will be discussed below. The baseline test can be uploaded to a Healthcare Service Provider, but as previously disclosed, is not read or reviewed until an injury is alleged. Before that time, there is no human interaction with the baseline test results. Only the proprietary software checks to insure all relevant/necessary test data is acquired.

Should an injury occur later to an individual who has previously been subjected to a baseline test, the injured individual returns for a post-loss test. The software determines protocols for the post-loss test which only evaluates a person's specifically injured body part/organ, which originates from the baseline test.

Upon receipt of the post-loss test results, the new post-loss test result data is compared with the stored baseline test result data to determine any differences between the data sets. If there are no differences, then the injured individual did not suffer an injury related to the description originally provided for the injured individual's position. In such a situation, there would likely be no compensable injury.

If there is a difference between the baseline test results and the post-loss test results, the difference between the two data sets is calculated and the difference between the baseline and post-loss test data objectively documents change in condition. Specific treatment recommendations may be made based on the documented difference(s) between the baseline test data and the post-loss test data.

The method is especially useful in a regulatory context, where failure to test each individual in a particular class results in discrimination issues. In the employment context, examples of classes could include employees in a company's shipping and receiving, in the mailroom, or in delivery. Also, by not reading/reviewing the test data, discrimination is prevented.

Each post-loss test protocol is directed to an individual's different muscle group that is related to the individual's job description. As a result, every muscle group involved in the job description is included in the final baseline test. For example, if the job description received is for a commercial truck driver, testing protocols that simulate that particular work environment are selected. The testing protocols will likely include a combination of protocols, such as range of motion (ROM), electromyography (EMG), functional capacity assessment (FCA), and pinch and grip strength. Typically, an electromyography (EMG) test is performed and monitored with a range-of-motion (ROM) test on an employee's hands, upper extremities, neck, shoulders, upper back, mid back, lower back, lower extremities and feet. Also, the EMG test performed and monitored with the ROM test and a functional capacity assessment (FCA) while an employee performs actions including lifting, pinching, gripping, carrying, reaching, climbing, pushing and pulling. The series of testing protocols may be generated by the server or a cloud, alone or in cooperation with a user.

Once the results of any given baseline test are stored, the data remains in storage until required for comparison against post-loss test results. Such a situation occurs, for example, when an employee that previously received a baseline test is injured while in a position fitting the designated job description and seeks worker's compensation for the injury.

A baseline test specific to a job description is broken up into different sections that address different body parts to generate the post-loss test. Each section includes a subset of testing protocols affiliated with the body part(s) that include the regions of injury on/in the employee. For example, if an employee suffers a neck injury, only those testing protocols that address the soft tissues of the neck, including discs, tendons, muscle pathology, and nerves (indirectly) will be included in the post-loss test. The stored data from the baseline test and post-loss test may be analyzed for any trends. This may be accomplished, for example via the proprietary software.

As previously disclosed, the post-loss test is generated from among the set of testing protocols included in the baseline test. The particular protocols selected to constitute the post-loss test are related to the specific region(s) of injury on/in the employee. The post-loss test results at the end of testing are then uploaded.

The method is not a library of measures, is not only a functional capacity assessment, does not just look for athletic injuries, and does not compare an assessment against functional jobs or tracking employees in an organization or evaluating a selected group of employees against a specific skill. The method compares pre and post-loss data and is a recommended best practice to assist in determining if an injury arises out of the course and scope of employment (AOECOE).

The method utilizes triggers for a baseline job-specific test and for new and existing employees, but the acquired data is not read/reviewed so no analysis or recommendations made have a specific event that triggers a post-loss test. Data is only encrypted to facilitate a data transfer.

The age of an injury is specific so it is not possible to perform pre and post comparison to determine the age of injury. The method compares a person to themself with specific body information that is only read/reviewed and compared to an individual's own test that is job specific. When a specific post-loss event is triggered it is matched in a database. The baseline test is only read/reviewed by use of the proprietary software.

It should be noted that the method can utilize various computer system configurations, including multi-core multiprocessor systems, minicomputers, mainframe computers, computers linked or clustered with distributed functions, as well as pervasive or miniature computers that may be embedded into virtually any device such as a smartphone or tablet.

Additionally, the computer/software can extrapolate the actions, including lifting, gripping and pinching, as well as muscle angle movement, from a baseline test without utilizing physiological data. The method does not determine if a person can perform the actions, thereby insuring that the method is compliant with relevant laws.

While the invention has been described in detail and pictorially shown in the accompany drawings it is not to be limited to such details, since many changes and modifications may be made to the invention without departing from the spirit and the scope thereof. Hence it is described to cover any and all modifications and forms which may come within the language and scope of the claims.

1. A method for determining change in condition for soft tissue injury, wherein said method determines if an injury occurs as a result of the course and scope of an individual's employment requirements or activities, wherein said method is designed to maintain objectivity in pre-loss and post-loss injury assessments by utilizing a non-loading and non-invasive functional assessment that is facilitated by proprietary software, wherein said method comprises the assessment steps of:
a) contract with an employer,
b) have an employee sign a consent form,
c) receive at a computer server a job description from the employer for the employee in a specific job position,
d) perform a non-invasive baseline test based on the specific job description and an employee's specific body part(s),
e) receive and store the baseline test results,
f) validate the baseline test results by the software,
g) receive a report of an alleged work related injury from the employee,
h) perform a software selected post-loss test that matches the baseline test and is segregated for the employee's specific body parts,
i) receive and store the post-loss test results,
j) validate the post-loss test results by the software,
k) match the post-loss test results to the baseline test results, and
l) compare the post-loss test results with the baseline test results to determine if there are differences between the post-loss test results and the baseline test results and record a comparison result.

2. The method for determining change in condition for soft issue injury as specified in claim 1 wherein said proprietary software functions in combination with a computer.

3. The method for determining change in condition for soft issue injury as specified in claim 1 wherein if a work related injury is confirmed, said method determines and suggests an optimal treatment for the injury.

4. The method for determining change in condition for soft issue injury as specified in claim 1 wherein an electromyography (EMG) test is performed and monitored with a range-of-motion (ROM) test on an employee's hands, upper extremities, neck, shoulders, upper back, mid back, lower back, lower extremities and feet.

5. The method for determining change in condition for soft issue injury as specified in claim 4 wherein the EMG test is performed and monitored with the ROM test and a functional capacity assessment (FCA) while an employee performs actions including lifting, pinching, gripping, carrying, reaching, climbing, pushing and pulling.

6. The method for determining change in condition for soft issue Injury as specified in claim 5 wherein the computer extrapolates the actions and angle movement of the actions without considering physiological data, wherein the actions are utilized to acquire test results when an employee performs the actions, and not to determine if an employee can perform the actions, thereby precluding discrimination.

7. The method for determining change in condition for soft issue injury as specified in claim 1 wherein the steps prompt a person who is administering the tests, wherein said method is designed to insure that the relevant steps are administered correctly and that the person administering the steps des not access, utilize or review the test results.

8. The method for determining change in condition for soft issue injury as specified in claim 1 wherein the baseline test results remain in an un-read state until an injury is alleged by an employee, wherein when an injury is alleged, the test results are uploaded, validated and stored without interaction from any person.

9. The method for determining change in condition for soft issue injury as specified in claim 1 wherein said proprietary software utilizes the employee data to match the baseline test results to said post-loss test results, wherein said software selects the baseline test that corresponds to the related post-loss test.

10. The method for determining change in condition for soft issue injury as specified in claim 9 wherein only said proprietary software matches the baseline test results to the post-loss test results for the employee's specific body parts that pertain to the injury the employee claims to have experienced on the job, wherein said software provides a determination based on the specific employee's change in condition, specific injury(ies), and relevant body part(s).

11. The method for determining change in condition for soft issue injury as specified in claim 4 wherein during the EMG test, the ROM test and a functional capacity assessment (FCA), said proprietary software monitors packet size of the data, amplitude of the respective signal(s), and change(s) of frequency for the ROM to ensure all data is acquired correctly and received correctly.

12. The method for determining change in condition for soft issue injury as specified in claim 1 further comprising instructions for insurance carriers and employers pertaining to the use of and performing the steps of said method.

13. The method for determining change in condition for soft issue injury as specified in claim 1 wherein the baseline test results and post-loss test results are accessed and reviewed only after an injury is alleged.

14. The method for determining change in condition for soft issue injury as specified in claim 13 wherein the two tests results are accessed and reviewed by said proprietary software only.
